(19) United States
(12) Patent Application Publication
(10) Pub. No.: US 2009/0088827 A1
(43) Pub. Date: Apr. 2, 2009

(54) Lead assembly providing sensing or stimulation of spaced-apart myocardial contact areas

(75) Inventors: Bruce Tockman, Scandia, MN (US); Lili Liu, Maple Grove, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc, ST PAUL, MN (US)

(21) Appl. No.: 11/906,794

(22) Filed: Oct. 2, 2007

(51) Int. Cl. A61N 1/05; A61B 5/042

(52) U.S. Cl. 607/123; 600/374

(57) ABSTRACT

Lead assemblies and methods for sensing or stimulating a first myocardial contact area and a second myocardial contact area when implanted are discussed. A lead assembly includes a lead body having at least one preformed biased portion at an intermediate portion thereof and an unbiased portion disposed between the biased portion and the distal end thereof. A first electrode is located at the preformed biased portion and is arranged to provide sensing or stimulation to the first myocardial contact area. A second electrode is located on the lead body distal to, and spaced apart from, the first electrode and is arranged to provide sensing or stimulation to a distinct second myocardial contact area spaced apart from the first myocardial contact area. In an example, the lead assembly includes a second preformed biased portion at the distal end of the lead body. In another example, the lead assembly includes additional electrodes.

### TECHNICAL FIELD

This patent document pertains generally to medical assemblies and methods. More particularly, but not by way of limitation, this patent document pertains to lead assemblies and methods providing sensing or stimulation of spaced-apart myocardial contact areas.

### BACKGROUND

A cardiac therapy system can include a battery powered implantable medical device (“IMD”) and one or more lead assemblies for delivering stimulation pulses to a subject's heart. Current IMDs include electronic circuitry for determining the nature of an irregular rhythm, commonly referred to as an arrhythmia, and for timing the delivery of a stimulation pulse for a particular purpose. The IMD is typically implanted into a subcutaneous pocket made in the wall of the subject's chest or elsewhere. Insulated wire assemblies called lead assemblies attached on a proximal end to the IMD are routed subcutaneously from the pocket to the shoulder or neck where the lead assemblies enter a major vessel, such as the subclavian vein. The lead assemblies are then routed into the site of pacing, usually a single area of the heart. Electrodes on the lead assemblies provide the electrical connection between the IMD and the heart.

Some subjects require a therapy system having multiple electrode sites in different areas of the heart for detecting and correcting an abnormal heartbeat. In the past, a common practice for these subjects was to provide two separate lead assemblies disposed at distinct heart locations. For instance, a first lead assembly would be implanted at a first site, such as the right atrium, while at least a second lead assembly would be implanted at a second site, such as the left ventricle, spaced from the first site.

### Overview

The present inventors have recognized, among other things, that having two separate lead assemblies is undesirable for many reasons. Among these reasons is the complexity of, and time required for, the implantation procedure of two lead assemblies as compared to that of the procedure for implanting a single lead assembly. In addition, two lead assemblies may mechanically interact with one another after implantation resulting in dislodgement of one or both of the lead assemblies. In vivo mechanical interaction of the lead assemblies may also cause abrasion of the insulative layer along the lead body possibly resulting in electrical failure of one or both of the lead assemblies. Another problem is that as more lead assemblies are implanted in the heart, the ability to add lead assemblies is reduced restricting treatment options should a subject's condition change over time. Two separate lead assemblies can also increase the risk of infection and may result in additional health care costs associated with re-implantation and follow-up.

The present inventors have also recognized a need for a single pass lead assembly having separate electrodes or electrode pairs for sensing or stimulating at least two distinct areas of the myocardium, such as from a single heart chamber. The present inventors have further recognized an unmet need for effective, reliable electrode/myocardial tissue contact through utilization of a lead body shape.

Lead assemblies and methods for sensing or stimulating a first myocardial contact area and a second myocardial contact area, such as from the left ventricle, are discussed. In certain examples, a lead assembly includes a lead body having at least one preformed biased portion at an intermediate portion thereof and an unbiased portion disposed between the biased portion and the distal end thereof. A first electrode is located at the preformed biased portion and is arranged to provide sensing or stimulation to the first myocardial contact area. A second electrode is located on the lead body distal to, and spaced apart from, the first electrode and is arranged to provide sensing or stimulation to a distinct second myocardial contact area spaced apart from the first myocardial contact area. In an example, the lead assembly includes a second preformed biased portion at the distal end of the lead body. In another example, the lead assembly includes additional electrodes, such as third and fourth electrodes, near one or both of the first or second electrodes.

In Example 1, a lead assembly comprises a lead body extending from a proximal end to a distal end and having an intermediate portion therebetween, the lead body having at least one preformed biased portion at the intermediate portion and an unbiased portion disposed between the biased portion and the distal end; a connector at the proximal end of the lead body; and a first electrode and a second electrode, the first electrode located at the preformed biased portion and arranged to provide sensing or stimulation of a first myocardial contact area when implanted, the second electrode located on the lead body distal to, and spaced apart from, the second electrode and arranged to provide sensing or stimulation of a distinct second myocardial contact area spaced apart from the first myocardial contact area when implanted.

In Example 2, the lead assembly of Example 1 is optionally configured such that the first and second electrodes are spaced apart from each other by an amount that is sufficient to avoid phrenic nerve stimulation at one of the first or second electrodes when implanted, when phrenic nerve, diaphragmatic or thoracic muscle stimulation is observed at the other of the first or second electrodes under otherwise similar pacing conditions.

In Example 3, the lead assembly of at least one of Examples 1-2 optionally comprises a second preformed biased portion at the distal end of the lead body.

In Example 4, the lead assembly of Example 3 is optionally configured such that the second preformed biased portion includes a preformed radius of curvature constructed and arranged to urge at least one electrode thereon toward a myocardial wall when implanted.

In Example 5, the lead assembly of at least one of Examples 1-4 optionally comprises at least a third electrode located on the lead body near the second electrode, the second and third electrodes forming a distal bipolar electrode pair to sense or stimulate the second myocardial contact area.

In Example 6, the lead assembly of Example 5 optionally comprises a drug eluting region located adjacent one or both of the second and third electrodes, the drug eluting region constructed and arranged to provide a drug to the second myocardial contact area associated with the second and third electrodes.

In Example 7, the lead assembly of at least one of Examples 1-6 optionally comprises at least a fourth electrode located on the at least one biased portion, the first and fourth electrodes forming an intermediate bipolar electrode pair to sense or stimulate the first myocardial contact area.

In Example 8, the lead assembly of Example 7 optionally comprises a drug eluting region located adjacent one or both of the first and fourth electrodes, the drug eluting region constructed and arranged to provide a drug to the first myocardial contact area associated with the first and fourth electrodes.

In Example 9, the lead assembly of Example 1, comprising at least a third and a fourth electrode located on the at least one biased portion.

In Example 10, the lead assembly of Example 9, comprising at least a fifth electrode located on the lead body near the second electrode.

In Example 11, the lead assembly of at least one of Examples 1-10 is optionally configured such that the at least one preformed biased portion includes a preformed three-dimensional bias constructed and arranged to urge at least one electrode thereon toward a myocardial wall.

In Example 12, the lead assembly of at least one of Examples 1-10 is optionally configured such that the at least one preformed biased portion includes a preformed two-dimensional bias constructed and arranged to urge at least one electrode thereon toward a myocardial wall.

In Example 13, the lead assembly of at least one of Examples 1-12 is optionally configured such that the second electrode is spaced at least between 1 cm and 3 cm from the first electrode.

In Example 14, the lead assembly of at least one of Examples 1-12 is optionally configured such that the second electrode is spaced at least between 3 cm and 6 cm from the first electrode.

In Example 15, the lead assembly of at least one of Examples 1-14 is optionally configured such that a cross-sectional size of the lead body is between about 4 Fr and 6 Fr.

In Example 16, a method comprises forming a lead body having a proximal end, a distal end, and an intermediate portion therebetween, including forming at least one biased portion at the intermediate portion and forming an unbiased portion between the biased potion and the distal end; and forming distinct sensing or stimulation myocardial contact areas of the lead body, including, locating a first electrode at the biased portion and locating a second electrode distal to the first electrode; and electrically coupling a first conductor with the first electrode and a second conductor with the second electrode.

In Example 17, the method of Example 16 optionally comprises forming a biased portion at the distal end of the lead body, including forming a radius of curvature constructed and arranged to urge at least one electrode on the biased portion at the distal end of the lead body toward a myocardial wall when implanted.

In Example 18, the method of at least one of Examples 16-17 optionally comprises forming a distal electrode pair constructed and arranged to sense or stimulate a lower portion of the myocardium, below a medial line, when implanted, including locating a third electrode on the lead body near the second electrode.

In Example 19, the method of Example 18 optionally comprises forming an intermediate electrode pair constructed and arranged to sense or stimulate an upper portion of the myocardium, above the medial line, when implanted, including locating a fourth electrode with the biased portion.

In Example 20, a method comprises accessing a lead body having at least one preformed biased portion at an intermediate portion thereof and an unbiased portion disposed distal to the preformed biased portion, including accessing a first electrode that is located at the preformed biased portion against a first myocardial contact area adjacent a first portion of a coronary vessel and accessing a second electrode that is located on the lead body distal to, and spaced apart from, the first electrode against a wall of a second myocardial contact area adjacent a second portion of the vessel, the first and second myocardial contact areas providing distinct myocardial sensing or stimulation contact areas; and using at least one of the first and second electrodes for sensing or electrostimulation.

In Example 21, the method of Example 20 optionally comprises selectively communicating at least one electrical pacing signal with one of the first or second myocardial contact areas.

In Example 22, the method of Example 21 optionally comprises avoiding phrenic nerve stimulation at one of the first or second electrodes when phrenic nerve, diaphragmatic or thoracic muscle stimulation is observed at the other of the first or second electrodes under otherwise similar pacing conditions.

In Example 23, the method of at least one of Examples 21-22 optionally comprises avoiding a high stimulation threshold at one of the first or second electrodes when a high stimulation threshold is observed at the other of the first or second electrodes under otherwise similar pacing conditions.

In Example 24, the method of at least one of Examples 21-23 optionally comprises sequentially pacing the first myocardial contact area and the second myocardial contact area.

In Example 25, the method of at least one of Examples 20-24 optionally comprises accessing a third electrode that is urged against the wall of the second myocardial contact area.

In Example 26, the method of Example 25 is optionally configured such that accessing the second and third electrodes comprises accessing second and third electrodes that are urged against the wall of the second myocardial contact area by allowing a distally positioned preformed radius of curvature to assume its preformed shape.

In Example 27, the method of Example 25 is optionally configured such that accessing the second and third electrodes comprises accessing the second and third electrodes that are urged against the wall of the second myocardial contact area by wedging the second and third electrodes into the second portion of the vessel.

In Example 28, the method of at least one of Examples 20-27 optionally comprises accessing a fourth electrode that is urged against the wall of the first myocardial contact area.

In Example 29, the method of at least one of Examples 20-28 optionally comprises inserting the lead body into the coronary vessel, including placing a guidewire within a vessel intersecting the first and second myocardial contact areas.

In Example 30, the method of at least one of Examples 20-29 optionally comprises inserting the lead body into the coronary vessel includes inserting a stylet into a lead body lumen; and guiding the stylet through a vessel intersecting the first and second myocardial contact areas.

In Example 31, the method of at least one of Examples 20-30 is optionally configured such that accessing the first electrode that is urged against the wall of the first myocardial contact area and accessing the second electrode that is urged against the wall of the second myocardial contact area includes accessing the same vessel branch with the first electrode and the second electrode.

In Example 32, the method of at least one of Examples 20-31 is optionally configured such that accessing the first electrode that is urged against the wall of the first myocardial contact area and accessing the second electrode that is urged against the wall of the second myocardial contact area includes accessing a first vessel branch with the first electrode and accessing a second vessel branch with the second electrode, the second vessel branch being oriented at an acute angle to the first vessel branch.

The present single pass lead assemblies are easy to implant due to their small size, and provide an opportunity for reliable sensing or stimulation of at least two distinct myocardial contact areas from the left ventricle. By way of electrode switching within or between the two or more contact areas, a user is provided with an option to improve or maximize a desired combination of, among other things, enhanced cardiac function response, prolonging of IMD battery through lower stimulation thresholds, or avoidance of unintended stimulation of the phrenic nerve, diaphragm or thoracic muscle.

These and other examples, advantages, and features of the present assemblies and methods will be set forth in part in the following Detailed Description. This Overview is intended to provide an overview of subject matter of the present patent application. It is not intended to provide an exclusive or exhaustive explanation of the invention. The Detailed Description is included to provide further information about the present patent application.

### BRIEF DESCRIPTION OF THE DRAWINGS

In the drawings, like numerals may be used to describe similar components throughout the several views. Like numerals having different letter suffixes may be used to represent different instances of similar components. The drawings illustrate generally, by way of example, but not by way of limitation, various embodiments discussed in the present document.

FIG. 1 is a schematic view illustrating a cardiac therapy system and an environment in which the therapy system can be used.

FIG. 2A is a side view illustrating a lead assembly including a first myocardial contact area and a second myocardial contact area.

FIG. 2B is a side view illustrating another lead assembly including a first myocardial contact area and a second myocardial contact area.

FIG. 2C is a side view illustrating an intermediate portion of another lead assembly, the lead assembly including a first myocardial contact area.

FIG. 2D is a side view illustrating an intermediate and distal end portion of another lead assembly including a first myocardial contact area and a second myocardial contact area.

FIG. 3A is a cross-sectional view illustrating a lead assembly taken along a line proximal a first electrode, such as along line 3A-3A of FIG. 2A, for example.

FIG. 3B is a cross-sectional view illustrating a lead assembly taken along a line proximal a first electrode, such as along line 3B-3B of FIG. 2B, for example.

FIG. 4 is a side view illustrating a lead assembly including a first myocardial contact area, a second myocardial contact area, and at least one drug region adjacent a myocardial contact area.

FIG. 5 is a schematic view illustrating a lead assembly including a first myocardial contact area and a second myocardial contact area, and an environment in which the lead assembly can be implanted.

FIG. 6 is a schematic view illustrating another lead assembly including a first myocardial contact area and a second myocardial contact area, and an environment in which the lead assembly can be implanted.

FIG. 7 is block diagram illustrating portions of a cardiac therapy system, including one conceptual example of circuitry within an IMD.

FIG. 8 is a block diagram illustrating an example method of manufacturing a lead assembly including a first myocardial contact area and a second myocardial contact area.

FIG. 9 is a block diagram illustrating an example method of using a lead assembly including a first myocardial contact area and a second myocardial contact area.

### DETAILED DESCRIPTION

Lead assemblies represent the electrical link between a medical device, such as an IMD, and a subject's cardiac or other bodily tissue, which is to be sensed or stimulated. A lead assembly generally includes a lead body that contains one or more electrical conductors extending from a proximal end to an intermediate portion or distal end thereof. As discussed in certain examples herein, the proximal end of the lead body includes a connector terminal couplable with the IMD, while the intermediate and distal end portions of the lead body include at least a first and a second electrode for contacting distinct areas of the myocardium.

The efficacy and longevity of an IMD can depend, in part, on the performance and properties of the lead assembly(s) used in conjunction with the device. For example, various properties of a lead assembly and the electrodes thereon will result in a characteristic stimulation threshold. Stimulation threshold is the energy required in a stimulation pulse to depolarize or “capture” the cardiac or other bodily tissue to which the pulse is directed. A relatively low threshold can be desirable to reduce or minimize the current drawn from a battery of the IMD in delivering a stimulation pulse. Increasing or maximizing the useful life of the battery can extend the useful life of the IMD, thereby reducing the need to replace the implanted device.

A factor that can affect the stimulation thresholds pertains to the location of the electrodes relative to the subject's cardiac or other bodily tissue to be sensed or stimulated. The number of electrodes and inter-electrode spacing can also affect the stimulation thresholds. An electrode's ability to sense or stimulate the subject's cardiac or other bodily tissue can depend, in part, on the relative location of the electrode(s) within, on, or near such tissue and the interface therebetween.

Beyond affecting stimulation thresholds, the location of lead electrodes relative to the subject's cardiac tissue to be sensed or stimulated can determine whether an unintended portion of the anatomy (e.g., the phrenic nerve, diaphragm or thoracic muscle) is unintentionally stimulated and can also determine the effect of the stimulation. Recognizing these reasons and more, the present inventors have conceived lead assemblies and methods including spaced-apart electrodes that are configured to contact the myocardium at distinct contact areas, such as from the left ventricle. Depending on a user-desired balance of, for example, low stimulation thresholds, avoidance of phrenic nerve stimulation, or desired therapeutic response, one or more certain electrodes vector combinations including the spaced-apart electrodes can be used.

### EXAMPLES

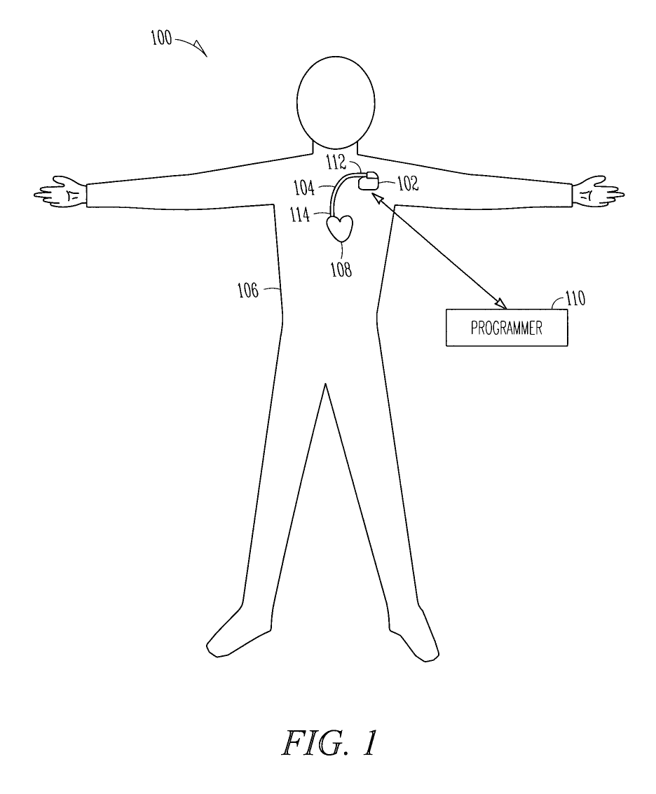

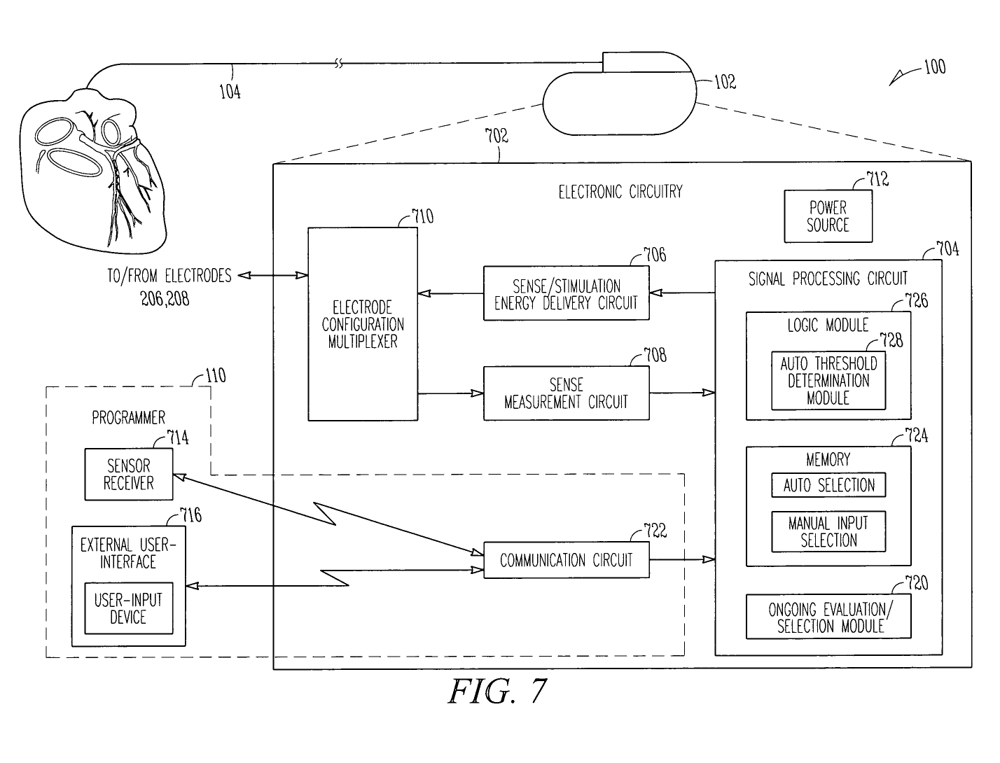

FIG. 1 illustrates a cardiac therapy system 100 and an environment in which the system may be used. The cardiac therapy system 100 can be used for receiving or delivering electrical signals or pulses to sense or stimulate, respectively, a heart 108 of a subject 106. As shown, the cardiac therapy system 100 can include an IMD 102, at least one lead assembly 104, and a local or remote external programmer 110. As discussed below, the lead assembly 104 includes electrodes to contact at least a first myocardial contact area and a distinct, second myocardial contact area. In an example, the IMD 102 is implanted in a subcutaneous pocket made in a wall of the subject's 106 chest, abdomen, or elsewhere. The lead assembly 104 connects with the IMD 102 on a proximal end 112 and with the heart 108 on a distal end 114, such that electronic circuitry 702 (FIG. 7) within the IMD 102 is in electrically communication with the heart 108.

The external programmer 110 and the IMD 102 are capable of wirelessly communicating data and instructions. In an example, the external programmer 110 and the IMD 102 use telemetry coils to wirelessly communicate data and instructions. Thus, the external programmer can be used to adjust the programmed therapy provided by the IMD 102, and the IMD 102 can report device data, such as battery or lead resistance, and therapy data, such as sensed and stimulation data, to the programmer 110 using telemetry. Optionally, the IMD 102 can be configured for electronically switching electrode vector combinations for sensing or stimulating the heart 108, as discussed in association with FIG. 7 below.

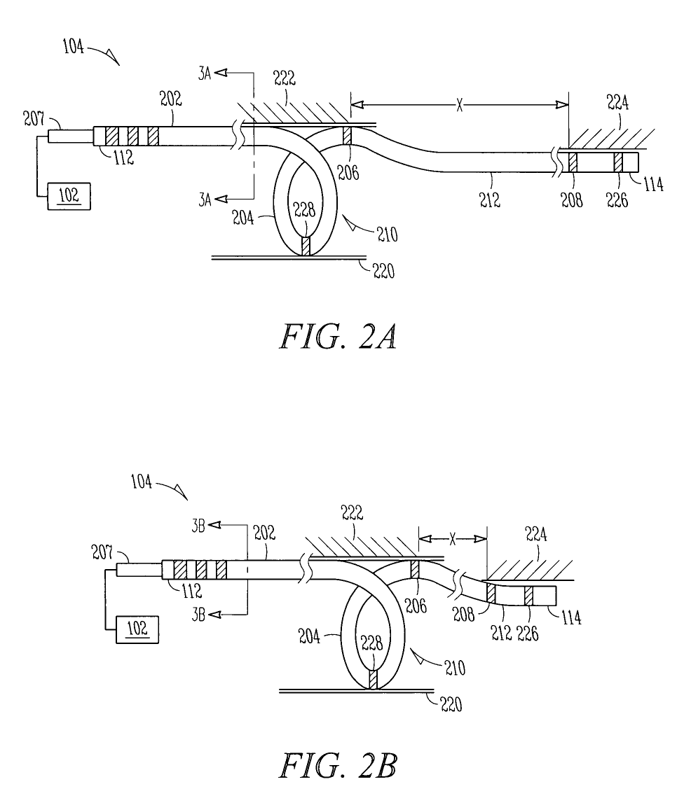

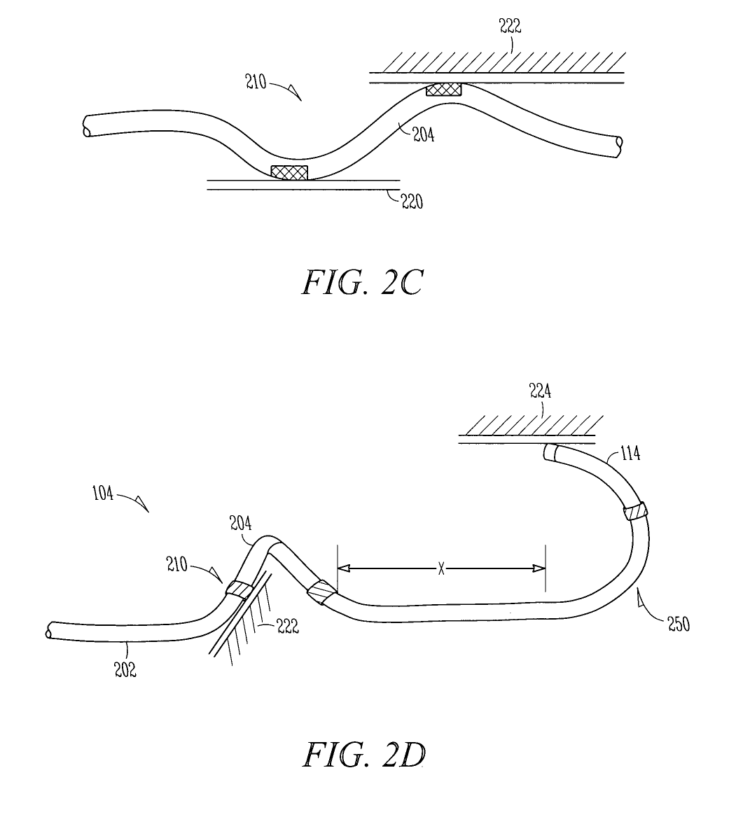

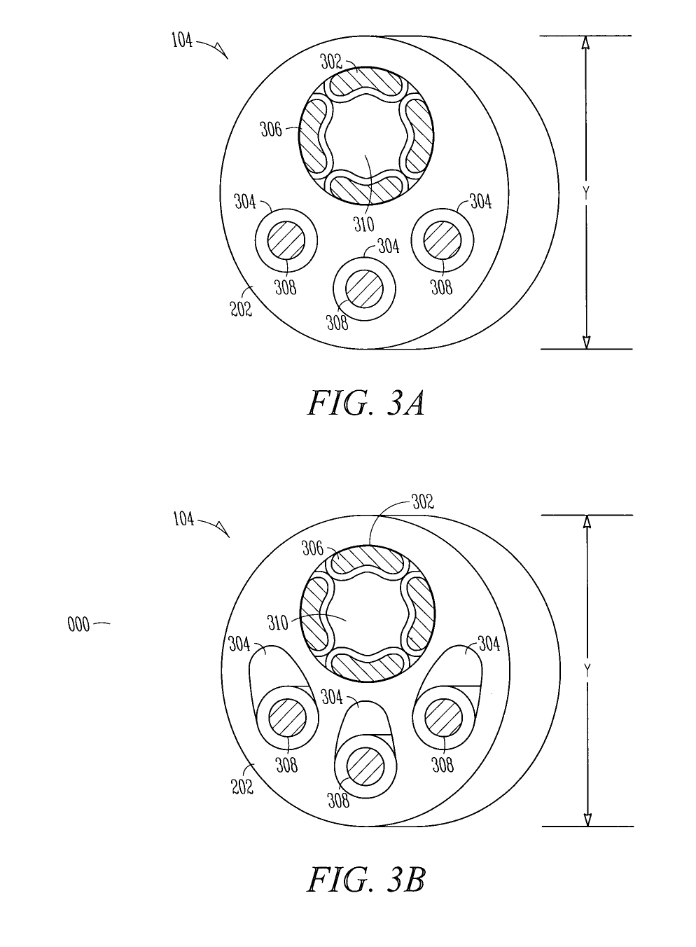

FIGS. 2A-2B are side views of two examples of a lead assembly 104. The lead assembly 104 includes a lead body 202 extending from a proximal end 112 to a distal end 114 and has an intermediate 204 portion therebetween. The lead assembly 104 is configured for implantation in coronary venous vasculature 220 of the heart 108 (FIG. 1) and for connection to an IMD 102 via a connector terminal 207. The connector terminal 207 is located at the proximal end 112 of the lead body 202 to electrically connect various lead electrodes and conductors (see FIGS. 3A-3B) disposed within the lead body 202 to the IMD 102.

The lead assembly 104 is constructed and arranged so that when implanted, at least a first electrode 206 and a second electrode 208 thereon are housed in the coronary venous vasculature 220 and urged into intimate contact with a vessel wall on the myocardial side and the left ventricle. To this end, the lead body 202 includes at least one preformed biased portion 210 at the intermediate portion 204 and an unbiased portion 212 disposed between the biased portion 210 and the distal end 114. The first electrode 206 is located at the preformed biased portion 210 and arranged to provide sensing or stimulation of a first myocardial contact area 222 when implanted. The second electrode 208 is located on the lead body 202 distal to, and spaced X apart from, the first electrode 206 and arranged to provide sensing or stimulation of a distinct second myocardial contact area 224 spaced from the first myocardial contact area 222 when implanted.

In the example of FIG. 2A, the spacing X between the first electrode 206 and the second electrode 208 can be between 3 cm and 6 cm, but not limited thereto. In the example of FIG. 2B, the spacing X between the first electrode 206 and the second electrode 208 can be between 1 cm and 3 cm, but not limited thereto. Optionally, the first 206 and second 208 electrodes are spaced X from each other by an amount that is sufficient to avoid phrenic nerve stimulation at one of the first or second electrodes, when phrenic nerve, diaphragmatic or thoracic muscle stimulation is observed at the other of the first or second electrodes under otherwise similar stimulation conditions. Optionally, the first 206 and second 208 electrodes are spaced X from each other by an amount that is sufficient to avoid a high stimulation threshold at one of the first or second electrodes when a high stimulation threshold is observed at the other of the first or second electrodes under otherwise similar pacing conditions. In an example, a pacing system analyzer (“PSA”) is used to determine the spacing X. In another example, use of a long or short spacing X depends on the anatomical conditions in which the lead assembly 104 will reside when implanted. For instance, if a subject 106 (FIG. 1) has a long vessel or side branch available form implantation, a lead assembly 104 having a longer spacing X can be chosen. If, on the other hand, the subject 106 only has a short vessel or side branch available for implantation, a lead assembly 104 having a shorter spacing X can be chosen.

The lead assembly 104 can also include at least a third 226 and a fourth 228 electrode. In an example, the third electrode 226 is located on the lead body near the second electrode 208, thereby forming a distal bipolar electrode pair to sense or stimulate the second myocardial contact area 224. In an example, the fourth electrode 228 is located near the first electrode 206, such as on the preformed biased portion 210, thereby forming an intermediate bipolar electrode pair to sense or stimulate the first myocardial contact area 222. Optionally, the lead assembly 104 can include alternative electrode placements along the lead body 202. For instance, in an example, the lead assembly 104 includes three independent electrodes on the preformed biased portion 210 and one electrode at the distal end 114. In another example, the lead assembly 104 includes three electrodes on the preformed biased portion 210, in which two or the three electrodes are electrically in common, and one electrode at the distal end 114. In another example, the lead assembly 104 includes three electrode on the preformed biased portion 210, in which two of the three electrodes are electrically in common, and two electrodes at the distal end 114. In each of these lead assembly 104 examples, the distal most electrode can be set back from the tip of the distal end 114 or can be a tip electrode.

While the lead assembly 104 can operate similarly to a bipolar lead assembly having positive (anodal) and negative (cathodal) portions of a circuit at discrete electrodes on the lead body 202, it should be noted that the lead assembly 104 can also operate in a unipolar mode. For instance, one or both electrodes of the electrode pairs can conjunctively act as the cathodal portion of the circuit, while the housing of the IMD 102 acts as the circuit's anodal portion. In another option, any of the electrodes not used as the cathodal portion of the circuit (including interlead combinations) can, alone or in combination, be used as the anode portion of the circuit. In yet another option, more than one electrode can be electrically tied together as the cathode portion of the circuit either within or outside of the electrode pairs.

The electrodes 206, 208, 226, 228 are of an electrically conductive material, such as an alloy of platinum and iridium, which is highly conductive and resistant to corrosion. Optionally, a surface of the electrodes 206, 208, 226, 228 is raised beyond the lead body 202. This raised surface arrangement can increase the chances of achieving intimate electrode/myocardial tissue contact thereby resulting in lower stimulation thresholds.

The at least one preformed biased portion 210 at the intermediate portion 204 of the lead body 202 is constructed and arranged to urge at least one electrode thereon toward a myocardial wall. In an example, the biased portion 210 has a three-dimensional bias, such as a helical shape with 1-2 turns. In another example, the biased portion 210 has a two-dimension bias, such as an S-shape or arch. The lead body 202 can be made of a biocompatible material having shape memory characteristics such that the biased portion 210 returns to its preformed shape once implanted and a stylet or guidewire is removed. An example of such a material is polyether polyurethane. In addition, the lead body 120 optionally has portions with shape memory characteristics, comprising either a shape memory polymer, a shape memory metal, or other materials treatable to retain a shape. Optionally, electrodes located on the biased portion 210 can be radially oriented about 120 degrees apart around a circumference of the lead body 202 to ensure myocardial contact of at least one electrode regardless of the lead assembly's orientation.

After implantation, the preformed biased portion 210 can be located in the coronary venous vasculature 220 such that at least one electrode 206, for example, abuts a first portion of the myocardium 222. The biased portion 210 can assist in maintaining the lead assembly 104 within the vessel 220 and can assist in enhancing electrode/myocardial tissue contact by one or more of the electrodes. As shown in the example of FIGS. 2A-2B, the helical biased portion 210 can be set back from the distal end 114 of the lead body 202, such as to reside in larger vessel diameter portions while placing the distal end 114 in narrower vessel portions. In alternative or in addition to helical or other three-dimensional bias configurations, two-dimensional shapes such as S-shapes, as shown in FIG. 2C, or arches can be used to enhance electrode/myocardial tissue contact.

As shown in FIG. 2D, the lead assembly 104 can optionally include a second preformed biased portion 250 at the distal end 114 of the lead body 202. Like the intermediate biased portion 210, the distal biased portion 250 can be constructed and arranged to urge at least one electrode thereon toward a myocardial wall, such as a second myocardial contact area 224, when implanted. In an example, the distal biased portion 250 includes a two-dimensional bias, such as a J-shape, to provide steerability and to help facilitate bias or fixation to the lead assembly 104. Optionally, a short straight tip can be added to the distal end 114, such as to serve as an atraumatic tip and to keep the lead body 202 from deflecting out of plane during implantation.

Advantageously, the present lead assemblies 104 can include separate electrode pairs, with each electrode pair contacting distinct areas of the myocardium. In addition, having at least one of the electrodes on a preformed biased portion helps position such electrode(s) against a vessel wall (e.g., a myocardial wall), thereby keeping stimulation thresholds to a minimum. In addition, the biased portion helps reduce dislodgement of the lead assembly 104. Although FIGS. 2A, 2B and 2D illustrate a lead assembly 104 including four electrodes, the present subject matter is not so limited. The lead assembly 104 may optionally include more or less than four electrodes, such as the alternative lead assemblies 104 discussed herein.

The cross-sectional views of FIGS. 3A-3B illustrate that the lead body 202 of the present lead assembly 104 can include one or more lumens, such as one coil-carrying lumen 302 and three cable-carrying lumens 304. In an example, the lead body 202 has an outer diameter Y of about 5 Fr. In another example, the lead body 202 has an outer diameter Y of about 4 Fr. In yet another example, the lead body 202 has an outer diameter Y of about 6 Fr. As shown, a coil conductor 306 is located in the coil-carrying lumen 302 and a cable conductor 308 is located in each of the three cable-carrying lumens 304. The conductors can be made of a highly conductive, highly corrosion-resistant material and can carry current and other signals between the IMD 102 (FIG. 1) and the electrodes. Optionally, at least one of the coil-carrying lumen 302 or the cable-carrying lumens 304 have a non-circular, space-saving shape as shown in FIG. 3B. This non-circular, space-saving shape can allow for easier stringing of conductors into and through the lead body 202 or allow for the creation of a smaller-sized lead body 202. For example, as the conductors are inserted, the shape of the lumens can deform to facilitate advancement of the conductors through the lead body 202. After the conductors are in place, the lumens can relax into their space-saving shape.

As discussed above, lead assemblies 104 including a preformed biased portion 210, 250 (see, e.g., FIG. 2D) can include a lumen into which a stylet or guidewire may be inserted. The stylet or guidewire provides a wire that can straighten out the lead body 202 while it is being implanted in a subject 106 (FIG. 1). By removing the stylet or guidewire, the lead body can take on its natural or preformed shape, such as a helical curve, S-shape or J-shape, for example. In an example, a lumen 310 formed by the coil conductor 306 can be used to receive the stylet or guidewire. Although FIGS. 3A-3B illustrate a quad-lumen lead body 202, the present subject matter is not so limited. The lead body 202 may optionally include more or less than four lumens, such as to accommodate one or more cable or coil conductors in any combination.

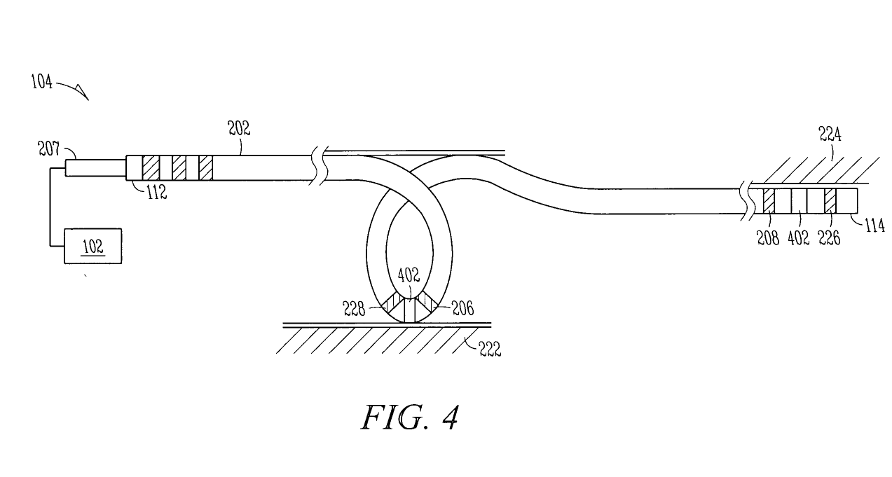

Referring to FIG. 4, a drug-providing (or other substance-providing) region (“drug region”) 402 is optionally located adjacent or contacting at least one electrode, such as to provide a desired amount of a drug to a first 222 or a second 224 myocardial contact area. The contents, structure, and size of the drug region 402 can vary depending on, among other things, the desired use of the drug region. As an example, the drug comprised in the drug region 402 can be one which is intended to counter thrombus formation, fibrosis, inflammation or arrhythmias, or any combination of drugs intended to accomplish one or more of these purposes, or any drug or combination of drugs intended to accomplish any other desirable localized purpose or purposes. As another example, the drug region 402 can be of any length or thickness to contain and apply the desired amount of drug to each electrode that it is near. As yet another example, the drug region 402 can be a separate element (e.g., a collar-like structure) secured to the lead body 202 or can be integrally molded into the lead body.

In an example, the drug region 402 comprises a carrier material and a drug. Typically, the carrier material is selected and formulated for an ability to incorporate the desired drug during manufacture and to release the drug within a subject 106 (FIG. 1) after implantation. The carrier material may comprise, among other things, silicone rubber or other polymer (e.g., polyurethane, polyethylene, ethylene-tetrafluoroethylene (ETFE), polytetrafluoroethylene (PTFE), or polyetheretherketone (PEEK)) or other material (e.g., metal or porous ceramics) that can hold or provide a drug. Alternatively, the carrier material may comprise a porous or non-porous material onto which a drug may be collated. The amount of any particular drug incorporated into the drug region 402 can be determined by the effect desired, the drug's potency, or the rate at which the drug capacity is released from the carrier material, or other factors.

In an example, the drug region 402 comprises a drug eluting matrix that elutes or otherwise provides a drug over time. In an example, the drug eluting matrix includes a steroid compounded with an uncured silicone rubber. Upon curing, the steroid becomes incorporated into a hardened polymeric binder. The curing process can be performed within a mold to produce a desired matrix shape. For instance, for a pacing lead, a rod or tube of dexamethasone acetate in silicone rubber can be cut to form a plug or ring.

In the example of FIG. 4, a first drug region 402 is located between, and shared by, the electrode pair 206/228 and a second drug region 402 is located between, and shared by, the electrode pair 208/226. The incorporation of a shared drug region in a lead assembly 104 can allow a desired electrode vector combination with an adjacent drug region to be chosen. It can also lower peak and chronic electrostimulation thresholds, such as by reducing inflammation or fibrotic growth. As discussed above, a reduction in stimulation thresholds increases the longevity of the IMD 102 because the current drain from the IMD's power source is reduced. In addition, a lead construction in which two or more electrodes share a drug region, such as a drug collar, can advantageously reduce or minimize an amount of drug needed on a per lead basis, resulting in cost savings. Optionally, a separate drug region 402 may be located adjacent or contacting each electrode.

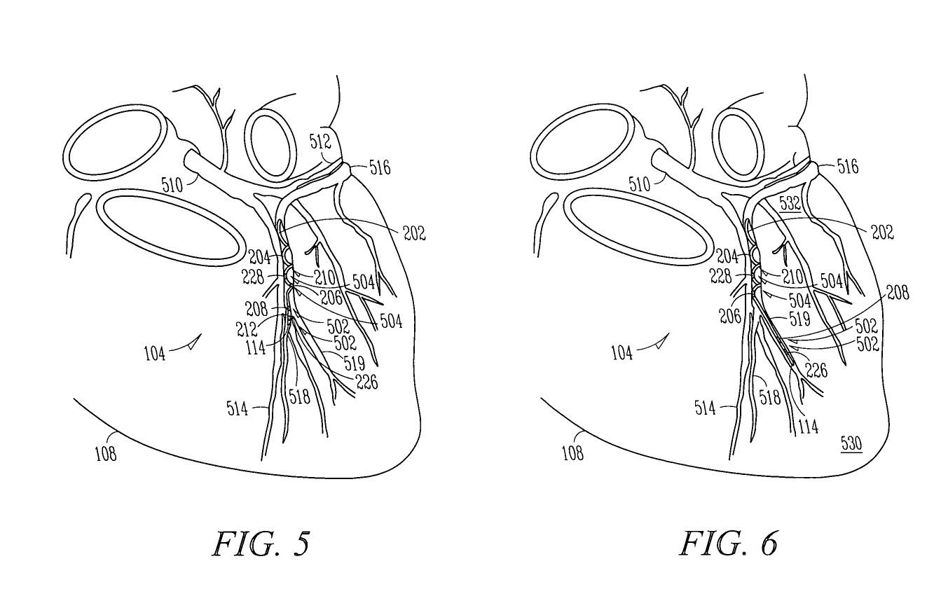

FIGS. 5-6 illustrate an intermediate portion 204 and distal end 114 of a lead body 202 disposed in a vein on the left ventricle of a subject's heart 108. Such example dispositions of a lead assembly 104, such as the lead intermediate portion 204 and distal end 114, are useful for sensing or delivering stimulation energy to a left side of the heart 108 for treatment of heart failure or other cardiac disorders needing therapy. The lead assemblies 104 shown include first 206, second 208, third 226, and fourth 228 electrodes, with the second and third electrodes forming a distal bipolar electrode pair 502 and the first and fourth electrodes forming an intermediate bipolar electrode pair 504. Due to variation in coronary venous anatomy, lead assemblies 104 having different inter-electrode pair spacing (e.g., the spacing between electrode pair 502 and the electrode pair 504) can be advantageous. For instance, the lead assembly 104 of FIG. 5 includes a shorter inter-pair spacing than the lead assembly 104 of FIG. 6 and thus, can be targeted for shorter primary cardiac vein branches or proximal secondary side branches. The longer inter-pair spacing found in the example of FIG. 6 can be targeted for distal placement in long primary vein branches or distal secondary side branches.

Whether long or short, the inter-electrode pair spacing of the present lead assemblies 104 advantageously allow distinctly different stimulation areas on the myocardium. The inter-electrode spacing allows a user to select a lead assembly 104 that matches a subject's anatomy. For instance, if a target implantation vein is long, the user (physician) can select a lead assembly 104 with longer inter-electrode pair spacing. This allows the lead assembly 104 to be implanted deep in the target vein with electrodes spaced X (FIG. 2A) far apart. If conditions in one region (e.g., near the distal electrode pair 502) are unfavorable (e.g., high stimulation thresholds or phrenic stimulation) the widely spaced intermediate electrode pair 504 can provide for distinctly different electrical performance. Alternatively, if the subject's available implantation anatomy is short, or if the target site is a proximal side branch, the user can select a lead assembly 104 with shorter inter-electrode pair spacing X (FIG. 2B) to fit the anatomy.

In addition to using the spacing for anatomical sizing, the inter-electrode pair spacing can be used to stimulate the distinctly different sites simultaneously or sequentially to either capture (or recruit) a larger area of the myocardium, or to affect the ventricular contraction pattern. Echocardiography or electrical mapping may provide a mechanical or electrical activation rationale for placing electrodes next to specific regions on the left ventricle to correct observed abnormalities. The lead assembly 104 with the most appropriate spacing X can be selected to address the observed abnormalities.

Referring to FIG. 5, the lead assembly 104 includes a preformed biased portion 210 on the intermediate portion 204 of the lead body 202 and an unbiased portion 212 disposed between the biased portion and the distal end 114. As shown, the left coronary artery 510 branches into the circumflex artery 512 and the anterior descending artery 514. The coronary sinus 516 branches into the coronary branch vein 518 and the coronary branch vein 519. Placing the intermediate and distal portions of the lead assembly 104 in the coronary vein 518 can be a suitable means for delivering stimulation therapy to subjects 106 (FIG. 1) suffering from congestive heart failure. As further shown, the biased portion 210 holds the electrode pair 504 thereon against the vessel wall in the larger, more proximal vessel locations, while the distal end 114 and electrode pair 502 thereon can be wedged in a smaller branch vessel. In an example, the first and second myocardial contact areas can be within the same main branch of a vessel and thus end up longitudinal to one another.

Referring to FIG. 6, the lead assembly 104 is configured for use within the coronary vein 518 and also within an acute coronary side branch vein 519 thereof. The intermediate portion 204 includes a preformed biased portion 210 including the intermediate bipolar electrode pair 504. The electrodes 206, 228 are shown in intimate contact with the vessel wall of the main coronary vein 518 where the electrodes contact a first myocardial contact area. The lead assembly further includes another bipolar electrode pair 502 distal to the intermediate portion 204. The electrodes 208, 226 can be wedged against a wall of the acute side branch vein 519 to contact a second myocardial contact area. The intimate contact between the electrodes and the myocardial tissue can help to reduce stimulation thresholds.

The biased portion 210 can be configured such that a coronary vessel of any size will reduce the diameter of the bias (e.g., helical bias) such that at least one electrode will be pressed against the myocardial wall. Optionally, additional electrodes can be strategically placed along the biased portion 210 increasing the probability of direct electrode contact with the myocardial wall of the vessel. As an example, multiple electrodes can be spaced apart along the biased portion 210 from the apex 530 to the base 532 of the heart 108. In another option, instead of pairs, single electrodes or more than two electrodes can be included.

In an example where multiple electrodes are connected to the same conductor, the electrode(s) with the best myocardial tissue contact can serve as the stimulation (cathode) electrode. In one example, the lead assembly 104 includes multiple electrodes and conductors, and the particular electrodes that act as the cathodes or anodes can be programmably or automatically selected, such as depending on the electrostimulation thresholds acquired at each stimulation site, depending on a cardiac resynchronization therapy (CRT) or other response, or another factor. In the examples of FIGS. 5-6, multiple electrode capacity is provided in the left ventricular vessels. The electrodes optionally can stimulate with a delay between them or sequentially. Although FIGS. 5-6 illustrate lead assemblies 104 having portions thereof located in an anterior vein, the present subject matter is not so limited.

FIG. 7 is a block diagram illustrating portions of a system 100 adapted to sense or stimulate (e.g., pace, defibrillate, or cardiovert) a heart 108 of a subject 106 (FIG. 1) at multiple locations within, on, or near the same. In the example shown, the system 100 includes a hermetically sealed medical device, such as an IMD 102, and an external programmer 110. The IMD 102 is connected to the heart 108 by way of at least one lead assembly 104. In varying examples, the at least one lead assembly 104 includes at least a first electrode 206 and a second electrode 208 arranged on a lead body to be urged into intimate contact with a first myocardial contact area and a distinct, second myocardial contact area, respectively.

Among other things, the IMD 102 can include a signal processing circuit 704, a sense/stimulation energy delivery circuit 706, a sense measurement circuit 708, an electrode configuration multiplexer 710, and a power source 712. Among other things, the external programmer 110 can include an external/internal sensor receiver 714 and an external user-interface 716 including a user-input device. The external/internal sensor receiver 714 can be adapted to receive subject specific information from one or more internal or external sensor(s).

The signal processing circuit 704 can be adapted to sense the heart 108 in a first instance and stimulate the heart in a second instance, each of which occur by way of a particular electrode vector combination selected from the at least two electrodes 206, 208 of the lead assembly 104 implanted within the subject 108 (FIG. 1) and one or more indifferent electrodes associated with the IMD 102. In an example, the signal processing circuit 704, can be programmed to automatically analyze various possible electrode vector combinations of the system 100 and select the one or more electrode vector combinations to be used in sensing or stimulating the heart 108. The IMD 102 can be further adapted (e.g., via an ongoing evaluation/selection module 720) to monitor and re-select the one or more electrode vector combinations as desired).

In another example, the programmer 110 can be programmed to automatically analyze various possible electrode vector combinations of the system 100 and select the one or more electrode vector combinations to be used in sensing or stimulating the heart 108. In yet another example, the one or more electrode vector combinations used to sense or stimulate the heart 108 can be selected manually by a caregiver (e.g., an implanting physician), and communicated to the IMD 102 using telemetry means associated with a communication circuit 722 of the IMD 102. In the example shown, such automatic or manual selection of the one or more electrode vector combinations can be stored in a memory 724. In yet another example, the one or more electrode vector combinations used to sense the heart 108 in a first instance and stimulate the heart in a second instance are the same. In a further example, the one or more electrode vector combinations used to sense the heart in a first instance and stimulate the heart in a second instance are different.

The one or more electrode vector combinations may be selected either automatically or manually using, at least in part, one or a combination of a stimulation threshold parameter, a stimulation selection parameter, or a heart chamber configuration parameter, for example. Other parameters that may be used to select the one or more electrode vector combinations are discussed in commonly assigned Hansen, U.S. patent application Ser. No. 11/230,989, entitled “MULTI-SITE LEAD/SYSTEM USING A MULTI-POLE CONNECTION AND METHODS THEREFOR,” which is herein incorporated by reference. In one example, at least one of the foregoing parameters are evaluated by way of a logic module 726 of the signal processing circuit 704 and is used in the selection of the one or more electrode vector combination used to sense or stimulation the heart 108.

In one example, a stimulation threshold parameter is used in the selection of the one or more electrode vector combinations for stimulating the heart 108. In varying examples, some or all possible electrode vector combinations are or can be evaluated to determine which one or more combinations optimally or acceptably use the lowest amount of output energy (e.g., stimulation pulse or shock) be applied to the heart 108 for capturing of the same.

Advantageously, by providing a system 100 adapted to determine which one or more electrode vector combinations use the lowest amount of energy while still ensuring reliable capture of the heart 108, the life of the IMD 102 may be prolonged, thereby reducing or minimizing the risk and expense to the subject 106 (FIG. 1) associated with early explantation and replacement of the IMD. In an example, the system 100 includes an autothreshold determination module 728 adapted to automatically determine whether a stimulation pulse or shock delivered through a first electrode vector combination has evoked a desired response from the heart 108, and if not, testing a second, third, . . . , etc. electrode vector combination for the desired heart response.

In another example, a stimulation selection parameter is used in the selection of the one or more electrode vector combinations for stimulating the heart 108. In varying examples, some or all possible electrode vector combinations are or can be evaluated to determine which one or more combinations optimally or acceptably provide appropriate therapy to the heart 108 while reducing, inhibiting, minimizing, or avoiding phrenic nerve, diaphragmatic or thoracic muscle stimulation. Advantageously, by providing a system 100 adapted to determine which one or more electrode vector combinations provides an appropriate balance between pulse or shock stimulation to the heart 108, while reducing, inhibiting, minimizing, or avoiding phrenic nerve, diaphragmatic or thoracic muscle stimulation ensures the subject 106 does not experience undesirable side effects.

In yet another example, a heart chamber configuration parameter is used in the selection of the one or more electrode vector combinations for stimulating the heart 108. In varying examples, some or all possible electrode vector combinations are or can be evaluated to determine which one or more combinations optimally or acceptably allow for sequential or multi-site stimulation of the heart such as for obtaining a desired hemodynamic response. In still another example, a spatial distance parameter is used in the selection of the one or more electrode vector combinations for stimulating the heart 108.

As illustrated in the example of FIG. 7, the IMD 102 may include the sense/stimulation energy delivery circuit 706 and the sense measurement circuit 708 to sense intrinsic or responsive activity of (e.g., in the form of sense indication signals), and provide stimulation to, the heart 108, respectively. In such an example, but not by way of limitation, the sense/stimulation energy delivery circuit 706 delivers a pacing pulse stimulation via a lead assembly 104 to one or more electrodes 206, 208 located on the left ventricle of the heart 108.

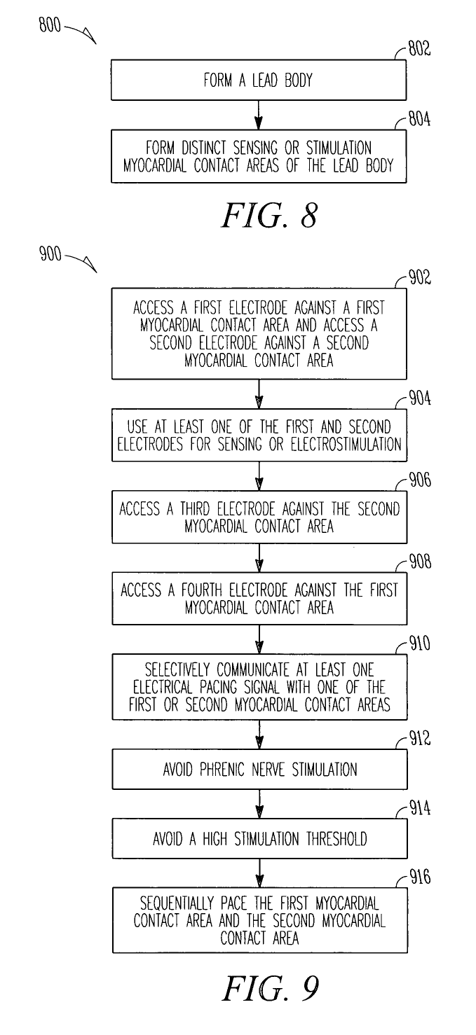

FIG. 8 is a block diagram illustrating an example method 800 of manufacturing a lead assembly including first and second myocardial contact areas, which are spaced apart from one another. At 802, a lead body is formed. The lead body extends from a proximal end to a distal end and has an intermediate portion therebetween. The intermediate portion includes at least one biased portion. In an example, the biased portion includes a two-dimensional bias. In another example, the biased portion includes a three-dimensional bias. The lead body further includes an unbiased portion between the biased portion and the distal end.

At 804, distinct sensing or stimulation myocardial contact areas are formed on the lead body. In an example, a first electrode is located at the biased portion and a second electrode is located distal to, and spaced from, the first electrode. When a conductor is electrically coupled to the first electrode and the second electrode, a first myocardial contact area served by the first electrode can be sensed or stimulated separated from a second myocardial contact area served by the second electrode, (or vice-versa).

In an example, a distal electrode pair is formed by the addition of a third electrode located on the lead body near the second electrode. This distal electrode pair can be used to sense or stimulate a lower portion of the myocardium, below a medial line, when implanted. In such an example, the distal electrode pair can be used to sense or stimulate a relatively posterior portion of the left ventricle. Optionally, a second biased portion can be formed at the distal end of the lead body to urge at least one electrode thereon toward the second myocardial contact area when installed. In another example, an intermediate electrode pair is formed by the addition of a fourth electrode located the lead body near the first electrode. This intermediate electrode pair can be used to sense or stimulate an upper portion of the myocardium, above a medial line, when implanted. In such an example, the intermediate electrode pair can be used to sense or stimulate a relatively anterior portion of the left ventricle.

FIG. 9 is a block diagram illustrating an example method 900 of using a lead assembly including first and second myocardial contact areas, which are spaced apart from one another. At 902, a lead body having at least one preformed biased portion at an intermediate portion thereof and an unbiased portion disposed distal to the preformed biased portion is accessed. By way of such accessing, a first electrode that is located at the preformed biased portion is accessed against a first myocardial contact area adjacent a first portion of a coronary vessel. In addition, a second electrode that is located on the lead body distal to, and spaced from, the first electrode is accessed against a wall of a second myocardial contact area adjacent a second portion of the vessel. This second myocardial contact area is distinct from the first myocardial contact area. In an example, the first electrode is accessed against a myocardial contact area adjacent a first vessel branch site and the second electrode is accessed against a myocardial contact area adjacent a second vessel branch site. In another example, the second vessel branch site is oriented at an acute angle to the first vessel branch. At 904, at least one of the first or second electrodes is used to sense or stimulate the first or second myocardial contact areas, respectively. At 906, a third electrode that is located near the second electrode is accessed against the second myocardial contact area. In this way, the second and third electrodes can form a distal bipolar electrode pair to sense or stimulate the second myocardial contact area independent of the first myocardial contact area. In an example, the second and third electrodes are accessed against the second myocardial contact area by allowing a distally positioned preformed radius of curvature to assume its preformed shape. In another example, the second and third electrodes are accessed against the second myocardial contact area by wedging such electrodes into the second portion of the vessel.

At 908, a fourth electrode that is located near the first electrode is accessed against the first myocardial contact area. In this way, the first and fourth electrodes can form an intermediate bipolar electrode pair to sense or stimulate the first myocardial contact area independent of the second myocardial contact area. In an example, the implantation of the intermediate or distal biased portions includes placing a guidewire within a vessel intersecting the first and second myocardial contact areas and threading the lead body thereover. In another example, the implantation of the intermediate or distal biased portion includes inserting a stylet into a lead body lumen and guiding the stylet though the vessel intersecting the first and second myocardial contact areas.

At 910, at least one electrical pacing signal is selectively communicated to at least one of the first or second myocardial contact areas. In an example, this selective communication includes comparing a hemodynamic response associated with the pacing signal at the first myocardial contact area to a hemodynamic response associated with the pacing signal at the second myocardial contact area. In another example, such as at 912, this selective communication includes avoiding phrenic nerve stimulation at one of the first or second electrodes when phrenic nerve, diaphragmatic or thoracic muscle stimulation is observed at the other of the first or second electrodes under otherwise similar pacing conditions. In another example, such as at 914, this selective communication includes avoiding a high stimulation threshold at one of the first or second electrodes when a high stimulation threshold is observed at the other of the first or second electrodes under otherwise similar pacing conditions. In yet another example, such as at 916, this selective communication includes sequentially pacing the first myocardial contact area and the second myocardial contact area in any order.

### CONCLUSION

Lead assemblies and methods for sensing or stimulating a first myocardial contact area and a second myocardial contact area when implanted are discussed. A lead assembly includes a lead body having at least one preformed biased portion at an intermediate portion thereof and an unbiased portion disposed between the biased portion and the distal end thereof. A first electrode is located at the preformed biased portion and is arranged to provide sensing or stimulation to the first myocardial contact area. A second electrode is located on the lead body distal to, and spaced apart from, the first electrode and is arranged to provide sensing or stimulation to a distinct second myocardial contact area spaced apart from the first myocardial contact area. In an example, the lead assembly includes a second preformed biased portion at the distal end of the lead body. In another example, the lead assembly includes additional electrodes, such as a third and fourth electrode, near one or both of the first or second electrodes.

The present single pass lead assemblies are easy to implant due to their small size, and provide an opportunity for reliable sensing or stimulation of at least two distinct myocardial contact areas. By way of electrode switching within or between the two or more contact areas, a user is provided with an option to improve or maximize a desired combination of, among other things, enhanced cardiac function response, prolonging of IMD battery through lower stimulation thresholds, or avoidance of unintended stimulation of the phrenic nerve, diaphragm or thoracic muscle.

Closing Notes

The above Detailed Description includes references to the accompanying drawings, which form a part of the Detailed Description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A,” and “A and B,” unless otherwise indicated. In this document, the phrase “implantable medical device” or simply “IMD” is used to include, but is not limited to, implantable cardiac rhythm management (CRM) systems such as pacemakers, cardioverters/defibrillators, pacemakers/defibrillators, biventricular or other multi-site resynchronization or coordination devices such as cardiac resynchronization therapy (CRT) device, subject monitoring systems, neural modulation systems, and drug delivery systems. In the appended claims, the terms “including” and “in which” are used as the plain-English equivalents of the respective terms “comprising” and “wherein.” Also, in the following claims, the terms “including” and “comprising” are open-ended, that is, a system, assembly, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine-implemented or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more features thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

What is claimed is:

1. A lead assembly comprising:
a lead body extending from a proximal end to a distal end and having an intermediate portion therebetween, the lead body having at least one preformed biased portion at the intermediate portion and an unbiased portion disposed between the biased portion and the distal end;
a connector at the proximal end of the lead body; and
a first electrode and a second electrode, the first electrode located at the preformed biased portion and arranged to provide sensing or stimulation of a first myocardial contact area when implanted, the second electrode located on the lead body distal to, and spaced apart from, the second electrode and arranged to provide sensing or stimulation of a distinct second myocardial contact area spaced apart from the first myocardial contact area when implanted.

2. The lead assembly of claim 1, wherein the first and second electrodes are spaced apart from each other by an amount that is sufficient to avoid phrenic nerve stimulation at one of the first or second electrodes when implanted, when phrenic nerve, diaphragmatic or thoracic muscle stimulation is observed at the other of the first or second electrodes under otherwise similar pacing conditions.

3. The lead assembly of claim 1, comprising a second preformed biased portion at the distal end of the lead body, the second preformed biased portion including a preformed radius of curvature constructed and arranged to urge at least one electrode thereon toward a myocardial wall when implanted.

4. The lead assembly of claim 1, comprising at least a third electrode located on the lead body near the second electrode, the second and third electrodes forming a distal bipolar electrode pair to sense or stimulate the second myocardial contact area.

5. The lead assembly of claim 1, comprising at least a fourth electrode located on the at least one biased portion, the first and fourth electrodes forming an intermediate bipolar electrode pair to sense or stimulate the first myocardial contact area.

6. The lead assembly of claim 1, comprising a drug eluting region located adjacent one or both of the first and second electrodes, the drug eluting region constructed and arranged to provide a drug to the first or second myocardial contact areas.

7. The lead assembly of claim 1, comprising at least a third and a fourth electrode located on the at least one biased portion.

8. The lead assembly of claim 7, comprising at least a fifth electrode located on the lead body near the second electrode.

9. The lead assembly of claim 1, wherein the at least one preformed biased portion includes a preformed three-dimensional bias constructed and arranged to urge at least one electrode thereon toward a myocardial wall.

10. The lead assembly of claim 1, wherein the at least one preformed biased portion includes a preformed two-dimensional bias constructed and arranged to urge at least one electrode thereon toward a myocardial wall.

11. The lead assembly of claim 1, wherein the second electrode is spaced at least between 1 cm and 3 cm from the first electrode.

12. The lead assembly of claim 1, wherein the second electrode is spaced at least between 3 cm and 6 cm from the first electrode.

13. The lead assembly of claim 1, wherein a cross-sectional size of the lead body is between about 4 Fr and 6 Fr.

14. A method comprising:
accessing a lead body having at least one preformed biased portion at an intermediate portion thereof and an unbiased portion disposed distal to the preformed biased portion, including accessing a first electrode that is located at the preformed biased portion against a first myocardial contact area adjacent a first portion of a coronary vessel and accessing a second electrode that is located on the lead body distal to, and spaced apart from, the first electrode against a wall of a second myocardial contact area adjacent a second portion of the vessel, the first and second myocardial contact areas providing distinct myocardial sensing or stimulation contact areas; and
using at least one of the first and second electrodes for sensing or electrostimulation.

15. The method of claim 14, comprising selectively communicating at least one electrical pacing signal with one of the first or second myocardial contact areas.

16. The method of claim 15, comprising avoiding phrenic nerve stimulation at one of the first or second electrodes when phrenic nerve, diaphragmatic or thoracic muscle stimulation is observed at the other of the first or second electrodes under otherwise similar pacing conditions.

17. The method of claim 15, comprising avoiding a high stimulation threshold at one of the first or second electrodes when a high stimulation threshold is observed at the other of the first or second electrodes under otherwise similar pacing conditions.

18. The method of claim 15, comprising sequentially pacing the first myocardial contact area and the second myocardial contact area.

19. The method of claim 14, comprising accessing a third electrode that is urged against the wall of the second myocardial contact area.

20. The method of claim 19, wherein accessing the second and third electrodes comprises accessing second and third electrodes that are urged against the wall of the second myocardial contact area by allowing a distally positioned preformed radius of curvature to assume its preformed shape.

21. The method of claim 19, wherein accessing the second and third electrodes comprises accessing the second and third electrodes that are urged against the wall of the second myocardial contact area by wedging the second and third electrodes into the second portion of the vessel.

22. The method of claim 14, comprising accessing a fourth electrode that is urged against the wall of the first myocardial contact area.

23. The method of claim 14, comprising inserting the lead body into the coronary vessel includes inserting a stylet into a lead body lumen; and guiding the stylet through a vessel intersecting the first and second myocardial contact areas.

24. The method of claim 14, wherein accessing the first electrode that is urged against the wall of the first myocardial contact area and accessing the second electrode that is urged against the wall of the second myocardial contact area includes accessing the same vessel branch with the first electrode and the second electrode.

25. The method of claim 14, wherein accessing the first electrode that is urged against the wall of the first myocardial contact area and accessing the second electrode that is urged against the wall of the second myocardial contact area includes accessing a first vessel branch with the first electrode and accessing a second vessel branch with the second electrode,
the second vessel branch oriented at an acute angle to the first vessel branch.
